(19) United States
(12) Patent Application Publication
(10) Pub. No.: US 2025/0101368 A1
(43) Pub. Date: Mar. 27, 2025

(54) CULTURE MATERIAL AND CULTIVATION METHOD OF GANODERMA AMBOINENSE

(71) Applicant: RIWAY (SINGAPORE) PTE. LTD., Singapore (SG)

(72) Inventor: Boon-Hong Lim, Singapore (SG)

(21) Appl. No.: 18/413,084

(22) Filed: Jan. 16, 2024

(30) Foreign Application Priority Data
Sep. 26, 2023 (TW) 112136908

(51) Int. Cl. C12N 1/14; C12M 1/00; C12M 1/12

(52) U.S. Cl. CPC C12N 1/14; C12M 31/00; C12M 37/00; C12N 2500/34; C12N 2500/76

(57) ABSTRACT

Provided is a culture material and a cultivation method of Ganoderma amboinense, which are used to increase the concentration of Ganoderiol F in the Ganoderma amboinense extract. First, the culture material is prepared, 38˜42 parts by weight of corn cobs, 6.65˜7.35 parts by weight of wheat bran, 6.65˜7.35 parts by weight of rice bran, 19˜21 parts by weight of seaweed powder, 23.75˜26.25 parts by weight of sawdust, and 0.95˜1.05 parts by weight of sucrose are mixed evenly to obtain a fungi bag. Next, sterilization is performed, followed by inoculation of the fungi of Ganoderma amboinense into the fungi bag and subsequent cultivation. Finally, Ganoderma amboinense is harvested when the fruiting body of the Ganoderma amboinense grows to a stipe length of 20-25 cm, and spore powder has not been released.

### CROSS-REFERENCE TO RELATED APPLICATION

This application claims the priority benefit of Taiwan application serial No. 112136908, filed Sep. 26, 2023, the full disclosure of which is incorporated herein by reference.

### BACKGROUND

### Technical Field

The disclosure relates to a culture material and a cultivation method of fungi. More particularly, this disclosure relates to a culture material and a cultivation method of Ganoderma amboinense.

### Description of Related Art

Generally, Ganoderma varieties are mainly Ganoderma lucidum and Ganoderma tsugae, and their appearance is mostly semicircular or fan-shaped. However, the antler-shaped Ganoderma is the most precious and rare. Because of its antler-like appearance, it was named Ganoderma amboinense (antle). The color of Ganoderma amboinense when grown is mostly waxy, tending towards red or brown. When Ganoderma amboinense grows to a height of about 20 cm and the top is halo white, it is called Antler Velvet. At this time, Ganoderma amboinense has the strongest vitality and the highest nutritional content.

Ganoderiol is one of the active ingredients in Ganoderma amboinense. It has various physiological activities such as antioxidant, anti-inflammatory, immune regulation and anti-tumor. Ganoderiol is believed to have benefits for boosting the immune system, regulating cholesterol and blood pressure, anti-inflammatory, antioxidant, anti-tumor, anti-allergic, and promoting cardiovascular health.

Modern nutritional medicine pays more and more attention to the research of Ganoderiol. Many scientific researches are devoted to exploring its biological activity and health benefits, and looking for potential applications in various diseases.

### SUMMARY

Accordingly, how to develop a culture material and a cultivation method for Ganoderma amboinense that can increase the content of Ganoderiol extracted from Ganoderma amboinense is a problem yet to be solved in the relevant technical field.

Hence, in one aspect, this disclosure is directed to a culture material and a cultivation method of the Ganoderma amboinense for increase the concentration of Ganoderiol in extract of Ganoderma amboinense.

A culture material for the Ganoderma amboinense is provided. The culture material of the Ganoderma amboinense comprises 38˜42 parts by weight of corncob, 23.75˜26.25 parts by weight of wood chip, 19˜21 parts by weight of seaweed powder, 6.65˜7.35 parts by weight of wheat bran, 6.65˜7.35 parts by weight of rice bran, and 0.95˜1.05 parts by weight of sucrose.

According to an embodiment, the cultivation method of the Ganoderma amboinense for increase the concentration of Ganoderiol in extract of Ganoderma amboinense is also provided. The cultivation method comprises a culture material preparation step, a sterilization step, an inoculation step, a mycelium growth management step, a ganoderma cultivation management step, and a harvesting step.

In the culture material preparation step, the culture material of the Ganoderma amboinense described above is evenly mixed. Afterward, the culture material of the Ganoderma amboinense is packed and sealed into a bag to obtain a fungi bag. In the sterilization step, the fungi bag is placed in a sterilization chamber at a sterilization temperature for sterilization. Subsequently, the fungi bag is cooled to room temperature. In the inoculation step, a strain of the Ganoderma amboinense is inoculated onto the culture material of the Ganoderma amboinense in the fungi bag in a sterile inoculation room to produce a fungi strain package. In the mycelium growth management step, the fungi strain package is moved into a dry, ventilated, and light-free fungi room for cultivation, so that the fungi strain can germinate. In the ganoderma cultivation management step, the fungi strain package is moved into a closed culture room, then the fungi strain package is opened for cultivation to grow a fruiting body of Ganoderma amboinense. In the harvesting step, the Ganoderma amboinense is harvested when the fruiting body of the Ganoderma amboinense grows to a stipe length of 20-25 cm, and spore powder has not been released.

According to an embodiment, the sterilization temperature is 121˜123° C.

According to another embodiment, a time for the mycelium growth management step is 22 days. A temperature of the fungi room is controlled at 23-28° C. on days 1 to 10, and the temperature of the fungi room is controlled at 25-28° C. on days 11-22.

According to yet another embodiment, a temperature of the culture room in the Ganoderma cultivation management step is controlled at 25-28° C. A relative humidity of the culture room is maintained at about 90% by spraying water or watering on the ground. A light of the culture room is illuminated for 10 hours a day, and a light intensity is 2000-5000 lux (LX).

According to yet another embodiment, the spraying and watering are stopped 7 days before the Ganoderma amboinense matures.

According to yet another embodiment, the light is provided by a fluorescent lamp as a light source.

According to yet another embodiment, the cultivation room is ventilated for a period of time every day.

In the foregoing, the culture material and the cultivation method of the Ganoderma amboinense is used to increase the concentration of Ganoderiol F in extract of Ganoderma amboinense.

These and other features, aspects, and advantages of this disclosure will become better understood with reference to the following description and appended claims. It is to be understood that both the foregoing general description and the following detailed description are by examples and are intended to provide further explanation of this disclosure as claimed.

### BRIEF DESCRIPTION OF THE DRAWINGS

In order to make the above-described technology of this disclosure, as well as other aspects, features, advantages, and embodiments, more apparent and understandable, the description of the accompanying drawings is provided as follows.

FIG. 1 is a tandem mass spectrum of the 8 ppm Ganoderiol F standard sample according to one embodiment of this disclosure.

FIG. 2 is a concentration analysis table of Ganoderiol F according to one embodiment of this disclosure.

### DETAILED DESCRIPTION

In the following detailed description, for purposes of explanation, specific embodiments of this disclosure will be illustrated in conjunction with the following figures. It is advantageous for the persons skilled in the art to fully utilize and practice this disclosure without undue interpretation and experimentation. However, this disclosure should not be construed as being limited to the embodiments set forth herein. That is to say, in some embodiments of this disclosure, these practical details are not necessary but are included to illustrate the implementation of this disclosure.

As mentioned above, in order to extract more Ganoderiol components from the Ganoderma amboinense, especially the content of Ganoderiol F (the structural formula is reproduced below). this disclosure provides a culture material and a cultivation method of the Ganoderma amboinense, which are described in details below.

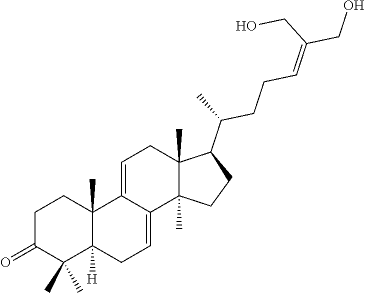

Culture Material of the Ganoderma amboinense

According to some embodiments of this disclosure, the culture material for the Ganoderma amboinense comprises 38˜42 parts by weight of corncob, 23.75˜26.25 parts by weight of wood chip, 19˜21 parts by weight of seaweed powder, 6.65˜7.35 parts by weight of wheat bran, 6.65˜7.35 parts by weight of rice bran, and 0.95˜1.05 parts by weight of sucrose. Among them, seaweed powder is a specially added ingredient to increase the Ganoderiol F content that may be extracted from the Ganoderma amboinense.

Cultivation Method of the Ganoderma amboinense

According to some other embodiments of this disclosure, the cultivation method of the Ganoderma amboinense is provided. The cultivation method comprises a culture material preparation step, a sterilization step, an inoculation step, a mycelium growth management step, a ganoderma cultivation management step, and a harvesting step. Details are provided below.

1. The culture material preparation step: The above-mentioned culture material of the Ganoderma amboinense is evenly mixed. Next, the culture material of the Ganoderma amboinense which is evenly mixed is packed and sealed (for example, install a collar and then cover it with a lid). Finally, a fungi bag may be obtained.

2. The sterilization step: After the fungi bag is placed in a sterilization chamber at a sterilization temperature for sterilization, the fungi bag is cooled to room temperature. The sterilization temperature may be 121˜123° C., the sterilization time may be 3 hours, and the pH value of the fungi bag after sterilization is 6.8˜7.5.

3. The Inoculation step: after the temperature of the fungi bag is cooled to room temperature (about 26-28° C.), the fungi of Ganoderma amboinense is inoculated into the culture material of the Ganoderma amboinense in the fungi bag in the sterile inoculation room. Then a fungi strain package may be obtained.

4. The mycelium growth management step: the fungi strain package is moved into a dry, ventilated, and light-free fungi room so that the fungi strain can germinate. A time for the mycelial growth management steps is 22 days. A temperature of the fungi room is controlled at 23-28° C. on days 1 to 10, and the temperature of the fungi room is controlled at 25-28° C. on days 11 to 22.

5. The Ganoderma amboinense management step: after the germinated fungi strain package is moved into a closed culture room, the fungi strain package is opened and cultured to grow the fruiting bodies of the Ganoderma amboinense. In the Ganoderma amboinense management step, a temperature of the culture room is controlled at 25-28° C. through an air conditioner outside the culture room. A relative humidity of the culture room is adjusted through a humidifier in the culture room by spraying water and watering on the ground to control the relative humidity at around 90%. The spraying and watering are stopped 7 days before the Ganoderma amboinense matures. A fluorescent lamps is used as the light source to illuminate for 10 hours a day, with a light intensity of 2000 to 5000 lux (LX).

The culture room needs to be ventilated for a period of time every day. This is because Ganoderma amboinense is an aerobic fungus and needs sufficient oxygen to promote the healthy growth of mycelium and avoid the formation of deformed Ganoderma amboinense. Therefore, as the mycelium of the Ganoderma amboinense grows, a ventilation time of the culture room needs to be increased in a timely manner to meet the demand for oxygen by Ganoderma amboinense. Ventilation in the culture room may be accomplished by adjusting the opening and closing of the vents in the culture room during the Ganoderma amboinense management step. The ventilation time of the culture room should be adjusted according to the temperature in the shed to ensure sufficient oxygen supply.

6. The harvesting step: the Ganoderma amboinense is harvested when the fruiting body of the Ganoderma amboinense grows to a stipe length of 20-25 cm and spore powder has not been released.

### Experimental and Comparative Examples

The Ganoderma amboinense is cultivated herein with the different culture material of the Ganoderma amboinense, and the concentration of Ganoderiol F in the samples is observed after extraction. The cultivation method and the extraction steps of the Ganoderma amboinense in Experimental example 1 and Comparative example 1 are the same. The difference is the composition of the culture material of the Ganoderma amboinense. In Experimental example 1, the culture material of the Ganoderma amboinense comprises 20 parts by weight of seaweed powder, compared to the culture material of Ganoderma amboinense in Comparative example 1, which used the conventional culture material and did not add any seaweed powder. Table 1 shows the detailed composition of the culture material of Ganoderma amboinense in Experimental example 1 and Comparative example 1.

TABLE 1
The culture material of Ganoderma Amboinense in
Experimental example 1 and Comparative example 1

| | Experimental example 1 | Comparative example 1 |
|---|---|---|
| Composition | (parts by weight) | (parts by weight) |
| corncob | 40 | 50 |
| wood chip | 25 | 35 |
| seaweed powder | 20 | — |
| rice bran | 7 | 7 |
| wheat bran | 7 | 7 |
| sucrose | 1 | 1 |

It may be seen the difference between the Experimental example 1 and Comparative example 1 from Table 1. The Experimental example 1 is different from Comparative example 1 by reducing approximately 10 parts by weight of corncob and 10 parts by weight of wood chip, and adding 20 parts by weight of seaweed powder. The main purpose is to increase the content of Ganoderiol F in the extract under the same extraction method.

The Ganoderma amboinense of Experimental example 1 and Comparative example 1 were extracted using the same extraction method. The steps of the extraction method are detailed below.

1. The Ganoderma amboinense is crushed with a grinder until the particle size is less than 0.25 cm.

2. Ganoderma amboinense powder is added into the 60% ethanol solvent, and a stirrer or stirring rod is used to stir the mixture of the mixture at a slow speed for 60 minutes.

3. The stirred mixture is placed in a constant temperature device and the appropriate temperature is set based on extraction requirements. Constant temperature extraction is performed for 60 minutes. During this process, the active ingredients of the Ganoderma amboinense are dissolved into the solvent.

4. A Buchner funnel and filter paper is prepared. The Buchanan funnel is placed over the collection container and a filter paper is placed in the funnel. The extract is poured gently and subjected by suction filtration. The filtrate is then collected.

5. The residue on the filter paper is mixed with 60% ethanol solvent, and the steps 3 and 4 are repeated.

6. The steps 3˜5 are repeated one more time.

7. The three filtrates is combined, concentrated under reduced pressure to remove the alcohol, and the extract of Ganoderma amboinense is frozen in a −80° C. environment.

8. The frozen extract of Ganoderma amboinense is placed under vacuum conditions so that the frozen liquid will directly sublime from the solid state to the gaseous state. The frozen water molecules are directly sublimated into water vapor to escape, thereby achieving the purpose of drying. The freeze-drying process can take hours or days, depending on the nature and quantity of the sample. The results obtained are listed in Table 2.

TABLE 2
The yield of the extract of Ganoderma Amboinense
of the Experimental example 1

| | Sample weight | EtOH | Extract weight | |
|---|---|---|---|---|
| Extraction times | (g) | (mL) | (g) | Yield (%) |
| 1 | 100.0 | 1,000 | 4.730 | 4.73 |
| 2 | 100.0 | 1,000 | 5.630 | 5.63 |
| 3 | 100.0 | 1,000 | 6.660 | 6.66 |

As may be seen from Table 2, the Ganoderma amboinense powder of Experimental example 1 was added to 1000 mL of 60% ethanol solvent, and then extracted according to the above steps. The extract obtained in the first extraction was 4.730 g, with a yield of 4.73%. For the second extraction, the Ganoderma amboinense powder was added to 1000 mL of 60% ethanol solvent for extraction. The weight of the extract for the second extraction was 5.630 g, and the yield was 5.63%. In the third extraction, after adding 1000 mL of 60% ethanol solvent to the Ganoderma amboinense powder, the extract weight of the third extraction was 6.660 g, and the yield was 6.66%.

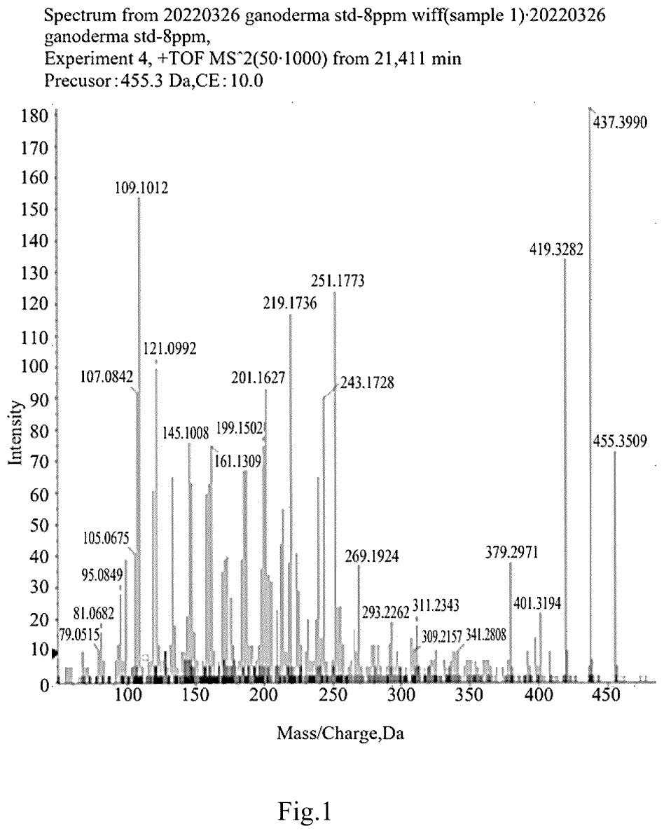

As shown in FIG. 1, the molecular weight of Ganoderiol F is 454.344, and the MS/MS peak of Ganoderiol F is m/z 455.35 (M+1 peak). Therefore, this disclosure indeed extracts Ganoderiol F from Ganoderma amboinense.

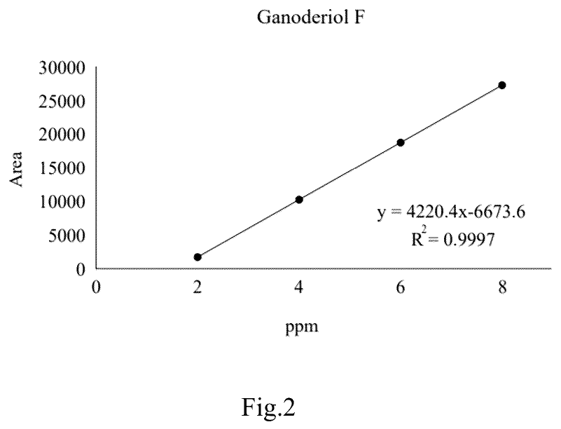

Standard samples of Ganoderiol F were prepared with concentrations of 2, 4, 6, and 8 ppm, and the amounts of the Ganoderiol F in the standard samples were quantified using a tandem mass spectrometer (MS/MS) to establish a calibration line for calculating the concentration of Ganoderiol F. The obtained integrated areas under the peaks and the concentrations of Ganoderiol F in the standard samples are listed in Table 3 below. Linear regression was performed utilizing the integrated areas under the Ganoderiol F peaks in the mass spectrum and the concentrations of the Ganoderiol F in the standard samples to obtain a regression equation: y=4220.4x+6673.6, R2=0.9997, as shown in FIG. 2.

TABLE 3
The concentration of Ganoderiol F standard sample and the integrated
areas of the spectral peaks in the mass spectrum.
Ganoderiol F standard sample

| | the integrated areas of the |
|---|---|
| concentration (ppm)(X-axis) | spectral peaks (Y-axis) |
| 2 | 1751.0275 |
| 4 | 10102.935 |
| 6 | 18907.137 |
| 8 | 26952.28964 |

Next, the samples of Experimental example 1 and Comparative example 1 were underwent MS/MS experimentations. The samples were prepared by the following method. After the extract of the Ganoderma amboinense fruiting body, obtained by the cultivation method described above, was dissolved into a solution, 10 mg of the solution was weighed and mixed with 1 mL methanol. Then, after centrifugation, 20 μl of the supernatant was taken and mixed with 80 μl ddH₂O (distillation-distillation H₂O)/0.1% FA (Formic Acid). Finally, 5 μl of the ddH₂O solution was taken and applied to a tandem mass spectrometer to measure the amount of Ganoderiol F from samples of Experimental example 1 and Comparative example 1.

It is known that the integrated peak area obtained from the sample of Experimental Example 1 was 20,334.695, and the peak area obtained from the sample of Comparative Example 1 was 8140.004. Then, the concentrations of Ganoderiol F in the extract of Ganoderma amboinense of Experimental example 1 and Comparative example 1 were calculated by using the regression equation to perform interpolation.

The integrated peak area in mass spectrum and the concentrations of Ganoderiol F in both Experimental example 1 and Comparative example 1 are listed in Table 4 below. From Table 4, the obtained concentration of Ganoderiol F in the extract of Ganoderma amboinense of Experimental example 1 is 6.40 ppm. The obtained concentration of Ganoderiol F in the extract of Ganoderma amboinense of Comparative example 1 is 3.51 ppm. Hence, the culture material described above in this disclosure can effectively increase the Ganoderiol F content in the extract of Ganoderma amboinense.

TABLE 4
Concentration of Ganoderiol F and peak area of sample
in Experimental example 1 and Comparative example 1.

| | Experimental | Comparative |
|---|---|---|
| | example 1 | example 1 |
| Integrated peak area | 20,334.695 | 8140.004 |
| Concentration of Ganoderiol F | 6.40 | 3.51 |
| (ppm) | | |

The variations and modifications made by persons skilled in the art should be viewed without departing from the scope of this disclosure.

What is claimed is:

1. A culture material of the Ganoderma amboinense, used to increase the concentration of Ganoderiol F in extract of Ganoderma amboinense, the culture material comprising:
38˜42 parts by weight of corncob;
23.75˜26.25 parts by weight of wood chip;
19˜21 parts by weight of seaweed powder;
6.65˜7.35 parts by weight of wheat bran;
6.65˜7.35 parts by weight of rice bran; and
0.95˜1.05 parts by weight of sucrose.

2. A cultivation method of the Ganoderma amboinense, used to increase the concentration of Ganoderiol F in Ganoderma amboinense extract, the cultivation method comprising:
a culture material preparation step for evenly mixing the culture material of the Ganoderma amboinense of claim 1, and packing and sealing the culture material of the Ganoderma amboinense into a bag to obtain a fungi bag;
a sterilization step for placing the fungi bag in a sterilization chamber at a sterilization temperature for sterilization, and cooling the fungi bag to room temperature;
an inoculation step for inoculating a strain of the Ganoderma amboinense onto the culture material of the Ganoderma amboinense in the fungi bag in a sterile inoculation room to produce a fungi strain package;
a mycelium growth management step for moving the fungi strain package into a dry, ventilated, and light-free fungi room for cultivation, so that the fungi strain can germinate;
a ganoderma cultivation management step for moving the fungi strain package into a closed culture room, then opening the fungi strain package for cultivation to grow a fruiting body of the Ganoderma amboinense; and
a harvesting step for harvesting the Ganoderma amboinense when the fruiting body of the Ganoderma amboinense grows to a stipe length of 20-25 cm, and spore powder has not been released.

3. The cultivation method of the Ganoderma amboinense of claim 2, wherein the sterilization temperature is 121˜123° C.

4. The cultivation method of the Ganoderma amboinense of claim 2, wherein a time for the mycelium growth management step is 22 days, a temperature of the fungi room is controlled at 23-28° C. on days 1 to 10, and the temperature of the fungi room is controlled at 25-28° C. on days 11-22.

5. The cultivation method of the Ganoderma amboinense of claim 2, wherein a temperature of the culture room in the Ganoderma cultivation management step is controlled at 25-28° C., a relative humidity is maintained at about 90% by spraying water or watering on the ground, a light is illuminated for 10 hours a day, and a light intensity is 2000-5000 lux (LX).

6. The cultivation method of the Ganoderma amboinense of claim 5, wherein the spraying and watering are stopped 7 days before the Ganoderma amboinense matures.

7. The cultivation method of the Ganoderma amboinense of claim 5, wherein the light is provided by a fluorescent lamp as a light source.

8. The cultivation method of the Ganoderma amboinense of claim 5, wherein the cultivation room is ventilated for a period of time every day.
